(19) United States
(12) Patent Application Publication
(10) Pub. No.: US 2012/0325222 A1
(43) Pub. Date: Dec. 27, 2012

(54) SKIN-TONE COLORED CONDOMS

(76) Inventor: Alesa J. Carr, Franklin, MI (US)

(21) Appl. No.: 13/528,111

(22) Filed: Jun. 20, 2012

Related U.S. Application Data
(60) Provisional application No. 61/499,566, filed on Jun. 21, 2011.

(51) Int. Cl. A61F 6/04

(52) U.S. Cl. 128/844

(57) ABSTRACT

The invention provides a condom made of latex with a skin tone appearance wherein said skin tone is selected from within a spectrum of skin tones. The invention also provides a condom array including plural skin toned condoms, said array suitable to allow a user to select a condom having a particular skin tone, said array comprising a plurality of condoms, each made of latex and each having a skin tone, at least one of said condoms having a different skin tone from another of the condoms.

### CROSS REFERENCE TO RELATED APPLICATION

This application claims the benefit of the filing date pursuant to the provisions of 35 U.S.C. §119(e), of the Provisional Patent Application Ser. No. 61/499,566, filed Jun. 21, 2011. the disclosures of the foregoing application is incorporated herein by reference.

### BACKGROUND OF THE INVENTION

It has long been recognized that, in addition to serving as an effective form of birth control, the use of condoms prevents the spread of sexually transmitted infections (STI's) such as the HIV virus, which causes AIDS. Considerable effort has been made to promote the adoption of condoms on numerous continents for developing countries as well as elsewhere, to prevent infection and unwanted pregnancies. However, the widespread adoption of condom use (both in the US and internationally) has been limited by a number of factors. One of the crucial factors is that, to date, condoms have not been manufactured with the particular skin-tone (or skin color or flesh tone) of the user in mind. Condoms are generally available in artificial colors as well as clear or gray. This fact has hampered the acceptance and use of condoms by people of color, who are put off by the existing clear or gray condoms.

### SUMMARY OF THE INVENTION

The invention provides a condom having the appearance of a skin-tone selected from a range of skin-tones where the range traverses a spectrum from lighter to darker. The availability of condoms with appearances corresponding to skin-tones within that range may help entice people to adopt more frequent condom use, many of whom would prefer a condom with an appearance of a skin tone to match their own skin. To the extent that personal or cultural distaste for condoms that differ from the user's own skin color contributes to a reluctance to use condoms, the availability of skin-toned condoms within that range may overcome this factor.

A potential user may be presented with an array of condoms, where condoms in the array have different colors or skin tones. The user may select a particular condom from the array based in the particular skin tone of the selected condom.

In accordance with one aspect, the invention provides a condom made of latex with the appearance of a skin tone wherein the skin tone is selected from the skin tones illustrated either in FIG. 1 or FIG. 2.

In accordance with another aspect, the invention provides a condom array including plural condoms, each having a skin tone, said array suitable to allow a user to select a condom having a particular skin tone, said array comprising a plurality of condoms, each made of latex and each having a skin tone, at least one of said condoms having a different skin tone from another of the condoms.

In accordance with still another aspect, the invention provides a condom array as stated above, where the skin tones of the condoms are selected from the skin tones illustrated in either FIG. 1 or FIG. 2.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The invention will now be described in the following portions of this specification when taken in conjunction with the attached drawings in which:

FIG. 1 shows an array of condoms having different skin tones including some with darker skin tones; and

FIG. 2 shows another array of condoms having different skin tones including some with darker skin tones.

### DETAILED DESCRIPTION OF PREFERRED EMBODIMENT

The invention relates to a condom with a particular skin tone, i.e. a skin tone selected from the colors shown in the attached drawings. Alternatively the invention provides an array of condoms having different skin tones. Preferably the different skin tones lie in a range of skin tones as illustrated herein. The condoms described herein comprise a natural rubber latex sheath in accordance with ASTM Latex Condom Standard D3492 or equivalent.

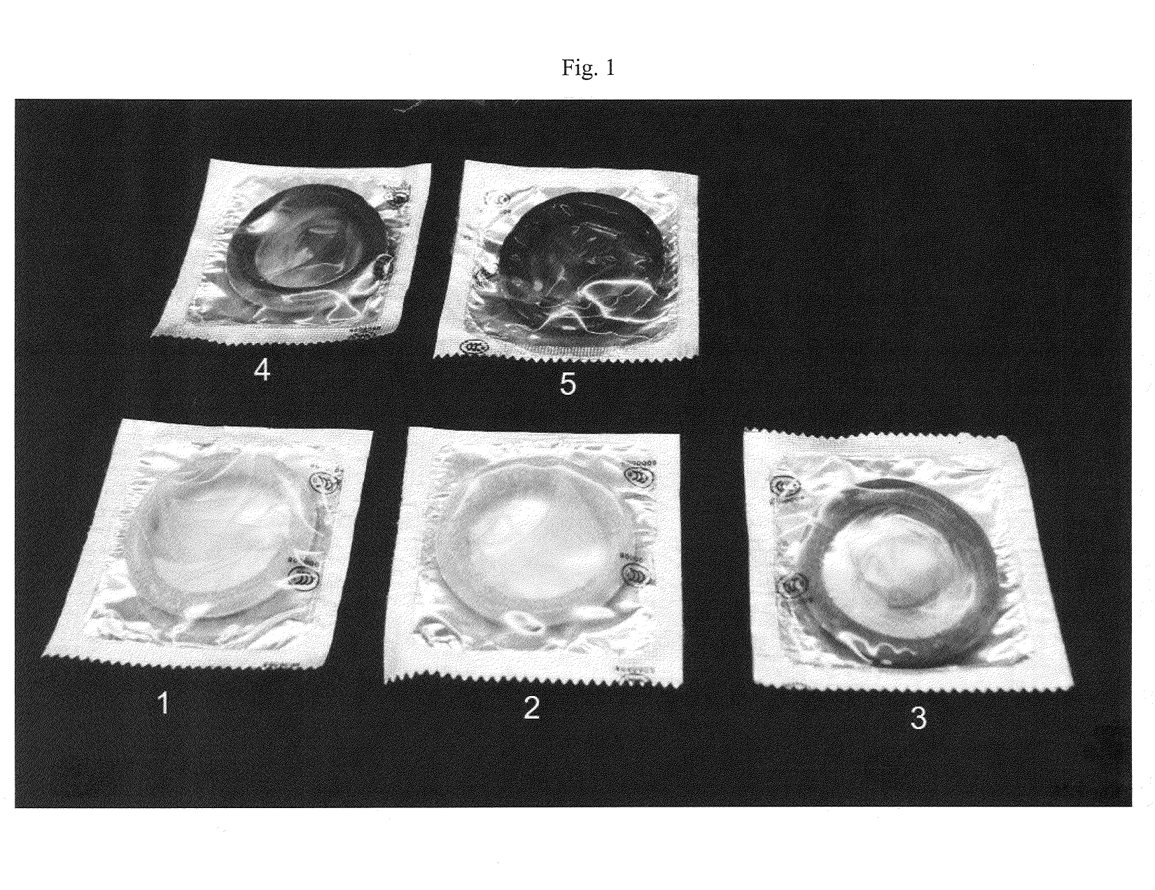

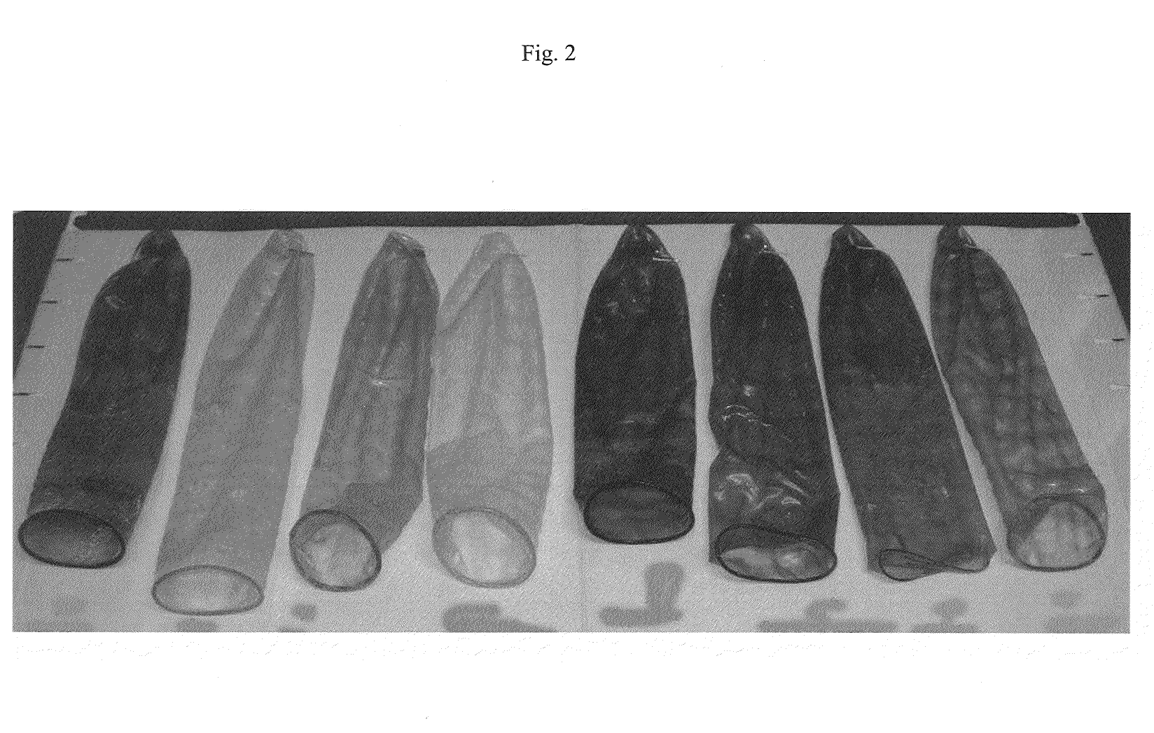

As shown in the attached photographs (FIG. 1 and FIG. 2), a range of different skin-tones are included in the array, from a very pale skin-tone color to a very dark skin-tone color, thereby allowing a wide range of skin colors to be matched.

The different colors or skin tones of the condoms illustrated herein are achieved by adding different pigments or mixtures of pigments to the latex.

FIG. 1 illustrates a first array of latex condoms with different skin tones. In particular the condoms labeled 1-5 illustrate skin-tones in a range of light to dark. FIG. 2 illustrates a different array of eight condoms. In the array of FIG. 2, the second and fourth condoms from the left show lighter skin-tones whereas the first, third and fifth through the eighth condoms from the left represent darker skin-tones. As is apparent each condom in the array of FIG. 1 has a different skin tone from each other condom. Likewise each condom in the array of FIG. 2 has a different skin tone from each other condom.

1. A condom made of latex with a skin tone appearance wherein said skin tone is selected from the skin tones illustrated in FIG. 1

2. A condom made of latex with a skin toned appearance wherein said skin tone is selected from the skin tones illustrated in FIG. 2.

3. A condom made of latex with a skin toned appearance wherein said skin tone is selected from the skin tones illustrated either in FIG. 1 or FIG. 2

4. A condom array including plural skin toned condoms, said array suitable to allow a user to select a condom having a particular skin tone, said array comprising a plurality of condoms, each made of latex and each having a skin tone, at least one of said condoms having a different skin tone from another of the condoms.

5. The condom array of claim 4 wherein the skin tones of the condoms are selected from the skin tones illustrated in FIG. 1.

6. The condom array of claim 4 wherein the skin tones of the condoms are selected from the skin tones illustrated in FIG. 2.

7. The condom array of claim 4 wherein the skin tones of the condoms are selected from the skin tones illustrated in either FIG. 1 or FIG. 2.
